(19) United States
(12) Patent Application Publication
(10) Pub. No.: US 2011/0086848 A1
(43) Pub. Date: Apr. 14, 2011

(54) Agrochemical Compositions Comprising Alkyl Polypropylene Glycol Polyethylene Glycol

(75) Inventors: Andreas STORK, Monheim (DE); Udo Reckmann, Koln (DE); Rolf Pontzen, Leichlingen (DE)

(73) Assignee: Bayer CropScience AG, Monheim (DE)

(21) Appl. No.: 12/879,758

(22) Filed: Sep. 10, 2010

Related U.S. Application Data
(60) Provisional application No. 61/242,119, filed on Sep. 14, 2009.

(30) Foreign Application Priority Data
Sep. 14, 2009 (EP) 09170160.7

(51) Int. Cl. A01N 43/88; A01N 43/40; C07C 43/04; A01P 7/00

(52) U.S. Cl. 514/229.2; 514/340; 514/341; 514/342; 568/613

(57) ABSTRACT

What is described are specific agrochemical compositions comprising alkyl polypropylene glycol polyethylene glycol, processes for their preparation and the use of corresponding compositions in the agrochemical field. The present invention furthermore relates to the use of alkyl polypropylene glycol polyethylene glycol as penetrant for agrochemically active compounds and/or as spontaneity enhancer in the preparation of application liquors.

The present invention relates to specific agrochemical compositions comprising alkyl polypropylene glycol polyethylene glycol, processes for their preparation and the use of corresponding compositions in the agrochemical field.

The present invention furthermore relates to the use of alkyl polypropylene glycol polyethylene glycol as penetrant or as spontaneity enhancer for agrochemically active compounds.

It is generally known that many agrochemically active compounds, in particular those having systemic action, have to penetrate into the plant so that their activity can unfold uniformly in the entire plant. Thus, when the active compounds are taken up via the leaves, the active compounds have to overcome the penetration barrier of the cuticles. It is furthermore of importance that the agrochemically active compounds penetrate into the plant quickly and distributed via a surface which is as large as possible, since there would otherwise be a risk of the active components being washed off by rain.

It is furthermore generally known that additives used in some crop protection compositions, such as, for example, surfactants, mineral oils and vegetable oils, promote the penetration of agrochemically active compounds into the plant and are thus able to increase the activity of the active compounds. Specifically, the additives may enhance wettability, improve adhesion of the spray droplets on the surfaces of the plants (=retention), lead to better distribution of the spray coating on the surface (=spreading) of the plant, increase the availability of the active compound in the dried spray residue by incipient dissolution or directly promote penetration of the active compound through the cuticles. Here, the additives are either incorporated directly into the formulation (which is possible only up to a limited percentage), or else added by the tank mix method to the application liquor in question.

Independently of the abovementioned properties of the additives, it is furthermore desirable for the additives to have advantageous properties when preparing the application liquor from a concentrate, i.e. even before the actual application to the plant. When preparing the application liquor, for example, high spontaneity is desirable, i.e. on introduction into water, for example, the concentrate should quickly and without major external aid distribute itself uniformly, without any crystals or oily substances separating off

The use of alkyl polypropylene glycol polyethylene glycol compounds in agrochemical compositions for achieving various effects is known from the prior art.

Thus, for example, WO 2008/077921 A1 discloses the use of a surfactant for preparing nanoparticles of water-insoluble active compounds in agrochemical formulations. In the formulations according to WO 2008/077921 A1, it is exclusively water-insoluble active compounds that are formulated in the form of nanoparticles. The surfactants to be used according to WO 2008/077921 A1 are preferably block copolymers of ethylene oxide units (C₂ building block) and alkylene oxide units have 3 to 10 carbon atoms.

This prior art neither uses water-soluble agrochemical compounds, nor is it indicated in the prior art how to improve penetration or spontaneity of agrochemical compositions.

WO 03/090531 A1 describes the use of certain alkoxylates as adjuvants for the agrochemical field. The alkoxylates are those which have a branched C₅- to C₃₀-alcohol moiety. Alkoxylates having a straight-chain alcohol moiety or having a branched alcohol moiety having up to four carbon atoms are not used in WO 03/090531 A1.

WO 00/42847 A1 relates to compositions consisting of a biocide component and an adjuvant component, the adjuvant component comprising at least surfactant from the group of specific alkoxylates. The alkoxylates described in WO 00/42847 A1 are all based on alcohol units having at least 6 carbon atoms.

WO 01/30147 A1 describes pesticide compositions and growth regulators for plants, insects or pests, comprising, as adjuvant, inter alia alkoxylates. The alkoxylates contain a terminal alkyl moiety based on higher alcohol homologs, for example a straight-chain C₁₂-C₁₄-alcohol. WO 01/30147 A1 does not disclose any alkoxylates based on lower alcohol homologs.

EP 0 356 812 A2 and WO 01/030147 A1, too, disclose agrochemical compositions comprising alkoxylates based on higher alcohol homologs.

Both with respective to enhancing penetration into the plant and with respect to enhancing spontaneity, these alkoxylates known from the prior art and based on higher alcohol homologs offer scope for improvement.

Accordingly, it is an object of the present invention to provide, based on the prior art illustrated above, further agrochemical preparations.

In a first embodiment, these agrochemical preparations and the application liquor prepared therefrom should preferably provide improved penetration of the agrochemically active compound comprised therein into the plants.

In a second embodiment, these agrochemical preparations should preferably have improved spontaneity on introduction into water for generating an application liquor.

In a third embodiment, these agrochemical preparations and the application liquors prepared therefrom should preferably simultaneously provide improved penetration of the agrochemically active compound comprised therein into the plants and have improved spontaneity of the preparation on introduction into water for generating an application liquor.

In a fourth embodiment, the improved spontaneity and/or penetration should not result in a simultaneous worsening of the other properties, in particular retention.

According to the invention, it has now been found that a composition comprising at least one active compound selected from the group consisting of compound B and compound A and at least one alkyl polypropylene glycol polyethylene glycol compound of the general formula (I)

R—O—(CₘH₂ₘO)ₓ—(CₙH₂ₙO)_{y}—R′ (I)

in which the individual radicals and indices have the following meanings:
R and R′ independently of one another represent hydrogen, a straight-chain C₁- to C₅-alkyl radical or a branched C₃- or C₄-alkyl radical;
m is 2 or 3;
n is 2 or 3;

x is from 5 to 150 and
y is from 5 to 150,
where one radical n or m has the meaning 2 and the other radical n or m has the meaning 3 can achieve the technical object.

Accordingly, the present invention firstly provides a composition comprising at least one active compound selected from the group consisting of compound B and compound A and at least one alkyl polypropylene glycol polyethylene glycol compound of the general formula (I)

R—O—(CₘH₂ₘO)ₓ—(CₙH₂ₙO)_{y}—R′ (I)

in which the individual radicals and indices have the following meanings:
R and R′ independently of one another represent hydrogen, a straight-chain C₁- to C₅-alkyl radical or a branched C₃- or C₄-alkyl radical;
m is 2 or 3;
n is 2 or 3;
x is from 5 to 150 and
y is from 5 to 150,
   where one radical n or m has the meaning 2 and the other radical n or m has the meaning 3.

In the context of the present invention, a straight-chain C₁- to C₅-alkyl radical is understood as meaning a methyl, ethyl, n-propyl, n-butyl or n-pentyl radical.

In the context of the present invention, a branched C₃- or C₄-alkyl radical is understood as meaning an isopropyl, isobutyl or tert.-butyl radical.

In a preferred embodiment, the radicals R and R′ are independently of one another selected from the group consisting of a methyl radical, an n-butyl radical and hydrogen.

In an even more preferred embodiment, the radicals R and R′ are independently of one another selected from the group consisting of an n-butyl radical and hydrogen.

With a view to the arrangement of the polyethylene and polypropylene moieties,
(a) either m has the value 2 and n has the value 3;
(b) or m has the value 3 and n has the value 2.

Preferred is embodiment (b) where m is 3 and n is 2.

Very particular preference is given to alkyl polypropylene glycol polyethylene glycol compounds of the formula (I) in which
m is 3,
n is 2,
x is from 5 to 80,
y is from 5 to 80,
R is n-butyl or hydrogen and
R′ is hydrogen.

According to the invention, it has been found that corresponding compositions generally have penetration-enhancing properties for water-soluble agrochemically active compounds.

According to the invention, it has furthermore been found that corresponding compositions generally increase spontaneity when preparing the application liquor from the concentrate.

According to the invention, it has furthermore been found that corresponding compositions generally have penetration-enhancing properties for water-soluble agrochemically active compounds and increase spontaneity when preparing the application liquor from the concentrate.

In the context of the present invention, the term “water-soluble agrochemically active compound” is generally understood as meaning an insecticidally and/or acaricidally active compound (i.e. an active compound that controls insects and/or Acarina) which has (at 20° C.) a solubility of ≧10 mg/l in water. Compounds A and B meet this requirement.

Suitable agrochemically active compounds which can be used in the composition according to the present invention are the active compounds below:

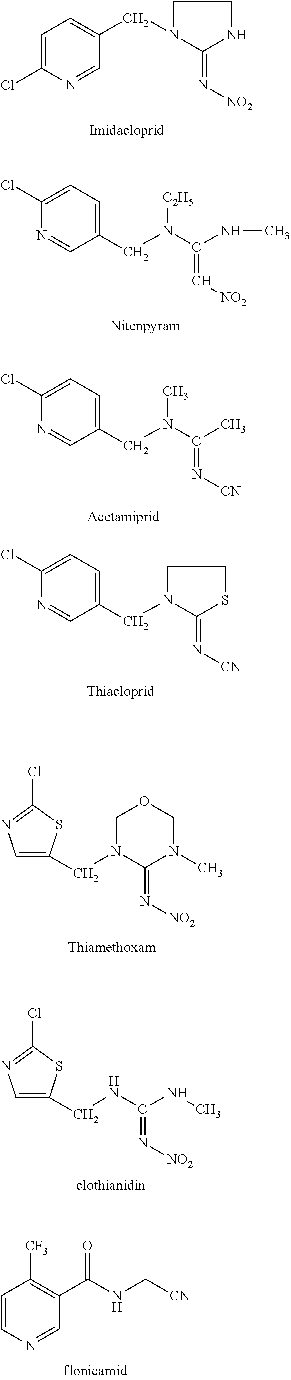

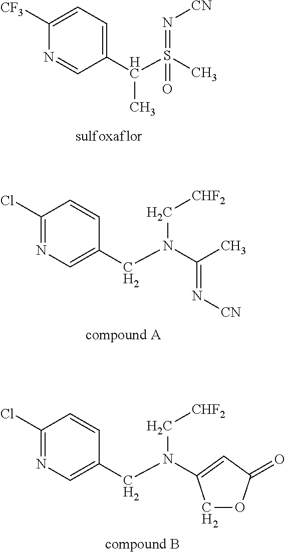

The compounds mentioned above and their use in the agrochemical field, for example as insecticides, are known.

Further insecticides or acaricides which, besides the agrochemically active compounds mentioned above, may additionally be present in the composition according to the invention are, for example, active compounds from the group of the pyrethroids, the phthalamides (flubendiamide), anthranildiamides (Cyazypyr, Rynaxypyr) or the ketoenol derivatives. The following compounds may be mentioned in an exemplary manner:

cypermethrin,

deltamethrin,

permethrin,

transfluthrin,

natural pyrethrum,

fenpropathrin,

cyfluthrin,

β-cyfluthrin and also

from the group of the ketoenol derivatives spirodiclofen, spiromesifen and spirotetramat.

These further insecticides or acaricides which may be present in the composition according to the invention as mixing partners with the at least agrochemically active compound can be either water-soluble or else water-insoluble.

Suitable fungicides which, besides the agrochemically active compounds mentioned above, may additionally be present in the composition according to the invention are active compounds, for example, from the group of the azoles, the strobilurin derivatives and the amino acid derivatives. The following compounds may be mentioned in an exemplary manner:

prothioconazole,

tebuconazole,

cyproconazole,

propiconazole,

triadimeriol,

myclobutanil,

trifloxystrobin,

azoxystrobin,

kresoxim-methyl,

maclostrobin,

fluoxystrobin,

iprovalicarb,

fluopicolide,

bixafen and

fluopyram,

The agrochemically active compounds mentioned above are known per se to the person skilled in the art (cf, Pesticide Manual; British Crop Protection Council; Edition: 14 (30 November 2006)).

These further fungicides, which may be present as mixing partners in the composition according to the invention, can be water-soluble or else water-insoluble.

In a further embodiment of the present invention, the composition generally comprises from 1 to 50% by weight, more preferably from 5 to 40% by weight, particularly preferably from 10 to 25% by weight, in each case based on the entire composition, of the water-soluble agrochemically active compound.

In the composition according to the invention, the alkyl polypropylene glycol polyethylene glycol compound of the general formula (I) is preferably used in a concentration of from 0.1 to 80% by weight, more preferably from 0.5 to 60% by weight, particularly preferably from 5 to 45% by weight, in each case based on the entire composition.

In a further embodiment of the present invention, the weight ratio of water-soluble agrochemically active compound to the alkyl polypropylene glycol polyethylene glycol compound of the general formula (is generally from 1:0.1 to 1:6, more preferably from 1:0.2 to 1:4, particularly preferably from 1:0.5 to 1:2.5. If in the present invention the weight ratio of water-soluble agrochemically active compound to the alkyl polypropylene glycol polyethylene glycol compound of the general formula (I) is in the range indicated above, a preferred balance between the effect of the alkyl polypropylene glycol polyethylene glycol compound of the general formula (I) and the costs associated with using this compound is achieved.

The use according to the invention of alkyl polypropylene glycol polyethylene glycol compounds of the general formula (I) has a number of advantages. Thus, these alkyl polypropylene glycol polyethylene glycol compounds are products which can be handled without any problems and which are also available in relatively large amounts. Moreover, they are biodegradable and allow a marked increase in effectiveness when applying water-soluble agrochemically active compounds against insects or Acarina and/or in the vector control associated therewith, and/or significant improvement of spontaneity.

Corresponding alkyl polypropylene glycol polyethylene glycols are commercially available and sold inter alia under the names Antarox® B/848, Antarox® B/500, Emulsogen® 3510, Emulsogen® EP 4901, Emulsogen® V 1816 or Genapol® PF 40.

The general formula (I) provides a general definition of the alkyl polypropylene glycol. polyethylene glycol compounds which can be used according to the invention. These alkyl polypropylene glycol polyethylene glycol compounds are generally mixtures of compounds of this type having various chain lengths. Accordingly, the indices x and y are average values in the ranges defined above which may also deviate from whole numbers.

In the context of the present invention, it is also possible to use mixtures of the alkyl polypropylene glycol polyethylene glycol compounds defined under the general formula (I), for example of two, three, four or five different alkyl polypropylene glycol polyethylene glycol compounds. If, in the context of the present invention, mixtures of a plurality of alkyl polypropylene glycol polyethylene glycol compounds are used, it is possible, for example, to select one alkyl polypropylene glycol polyethylene glycol compound such that the penetration of the agrochemically active compound is optimized and the other alkyl polypropylene glycol polyethylene glycol compound such that the spontaneity of the agrochemically active compound is optimized,

Furthermore, the composition according to the invention may generally comprise from 1 to 90% by weight, more preferably from 15 to 75% by weight, particularly preferably from 30 to 60% by weight, in each case based on the composition, of one or more solvents.

Suitable solvents which may be used in the context of the present invention are preferably water-soluble and/or water-miscible, solvents. Corresponding examples of suitable solvents are y-butyrolactone, dimethyl isosorbide, dimethyl sulfoxide (DMSO), propylene glycol, propylene carbonate, benzyl alcohol, dimethylamides, short-chain alcohols, in particular ethanol, n-propanol, isopranol, n-butanol, isobutanol and tert-butanol, N-methyl-2-pyrrolidone (NMP), polyethylene glycol, methoxy-2-propanol, triethylene glycol diacetate and water.

Further optional components of the composition according to the invention are, for example, additives in amounts of in general from 0 to 30% by weight, more preferably from 0 to 20% by weight, particularly preferably from 0 to 10% by weight. Additives which may be present in the crop treatment compositions according to the invention are to be understood as being further agrochemically active compounds, and also crystallilation inhibitors, wetting agents, emulsifiers and water.

Crystallization inhibitors which may be present in the compositions according to the invention are all compounds which can customarily be used for such purposes in agrochemical compositions. N-alkylpyrrolidones, such as N-octylpyrrolidone and N-dodecylpyrrolidone, may be mentioned as being preferred, furthermore copolymers of polyvinylpyrrolidone and polyvinyl alcohol, such as, for example, the polyvinylpyrrolidone/polyvinyl alcohol copolymer known under the name Luviskol VA 64 (from BASF), furthermore alkylcarboxylic acid dimethylamides, such as decanoic acid dimethylamide or the C₆₋₁₂-alkanecarboxylic acid dimethylamide mixture known under the name Hallcomid® (from. Hall Comp.), and furthermore copolymers of ethylenediamine with ethylene oxide and propylene oxide, such as, for example, the product known under the name Synperonic T 304 (from Uniqema).

Suitable wetting agents are all compounds which can customarily be used for such purposes in crop treatment compositions. Alkylphenol ethoxylates, alkylnaphthalenesulfonates, dialkyl sulfosuccinates, such as dioctyl sulfosuccinate sodium, lauryl ether sulfates and polyoxyethylene sorbitan fatty acid esters may be mentioned as being preferred.

For their part, the alkyl polypropylene glycol polyethylene glycol compounds to be used according to the invention and employed for improving penetration and/or for improving spontaneity have emulsifier properties. Nevertheless, in the context of the present invention, a further emulsifier may optionally be used in the composition according to the invention.

In this case, suitable emulsifiers are all customary nonionogenic, anionic, cationic and zwitterionic compounds having surface-active properties which are customarily used in agrochemical compositions. These compounds include reaction products of fatty acids, fatty esters, fatty alcohols, fatty amines, alkylphenols or alkylarylphenols with ethylene oxide and/or propylene oxide and/or butylene oxide, and also their sulfuric esters, phosphoric monoesters and phosphoric diesters, furthermore reaction products of ethylene oxide with propylene oxide, furthermore alkylsulfonates, alkyl sulfates, aryl sulfates, tetraalkylammonium halides, trialkylarylammonium halides and alkylaminesulfonates. The emulsifiers may be used individually or else as a mixture. Reaction products of castor oil with ethylene oxide in a molar ratio of from 1:20 to 1:60, reaction products of C₆-C₂₀-alcohols with ethylene oxide in a molar ratio of from 1:5 to 1:50, reaction products of fatty amines with ethylene oxide in a molar ratio of from 1:2 to 1:25, reaction products of 1 mol of phenol with 2 to 3 mol of styrene and 10 to 50 mol of ethylene oxide, reaction products of C₈-C₁₂-alkylphenols with ethylene oxide in a molar ratio of from 1:5 to 1:30, alkyl glycosides, C₈-C₁₆-alkylbenzenesulfonic acid salts, such as, for example, calcium, monoethanolammonium, diethanolammonium and triethanolammonium salts, may be mentioned by way of preference.

Examples of nonionic emulsifiers which may he mentioned are the products known under the names Pluronic PE 10 100 from BASF) and Atlox 4913 (from Uniqema). Also suitable are tristyrylphenyl ethoxylates. Examples of anionic emulsifiers which may be mentioned are the product commercially available under the name Baykanol SL (=condensate of sulfonated ditolyl ether and formaldehyde), and also phosphated or sulfated tristrylphenol ethoxylates, where specific mention is given to Soprophor ELK and Soprophor 4D 384 (from Rhodia).

The content of the individual components in the plant treatment compositions according to the invention may be varied within a certain range. Preference is given to plant treatment compositions in which the concentrations
of alkyl polypropylene glycol polyethylene glycol compounds of the general formula (1) are from 0.1 to 80% by weight;
of agrochemically active compound are from 1 to 50% by weight;
of solvents are from 1 to 90% by weight and
of additives are from 0 to 30% by weight.

The present invention furthermore relates to the preparation of the composition according to the invention. The crop treatment compositions according to the invention are generally prepared by mixing the individual components in the respective desired ratios. In general, one agrochemically active compound is initially charged, and the other components are then added with stirring, in any order.

When preparing the composition according to the invention, the temperatures may be varied within a certain range. In general, the operations are carried out at temperatures between 10 and 70° C., preferably at room temperature.

Suitable for preparing the composition according to the invention are customary apparatus used for preparing agrochemical formulations.

The compositions according to the invention can be applied either as such or after prior dilution with water or other diluents, i.e., for example, as emulsions, suspensions, solutions or aerosols. In the case of direct application, the composition according to the invention improves at least the penetration of the agrochemically active compound.

In a particularly preferred embodiment, the composition according to the invention is present in the form of a concentrate which is diluted with a solvent, in particular water, prior to the actual application. In this case, the composition according to the invention improves the penetration of the agrochemically active compound and/or the spontaneity of the preparation when preparing the application liquor.

In addition, the present invention also relates to the use of corresponding compositions in the agrochemical field. The present invention relates in particular to the use of the compositions according to the invention for controlling insects and/or Acarina. In the context of the use according to the invention, the composition according to the invention is generally initially introduced into an aqueous phase, which is then applied.

Here, application is carried out using customary methods, i.e., for example, by spraying, watering, atomizing, injecting or spreading.

The application rate of the plant treatment compositions according to the invention can be varied within a relatively wide range. It depends on the respective active compounds present and their concentration in the formulations.

With the aid of the plant treatment compositions according to the invention, it is possible to apply water-soluble insecticidally and/or acaricidally active compounds in a particularly advantageous manner to the plants and/or their habitat. Here, the penetration power of the active compounds is enhanced, the biological activity of the active compounds is increased compared to conventional formulations and/or the spontaneity of the products on introduction into water is improved.

The present invention furthermore relates to the use of alkyl polypropylene glycol polyethylene glycol compounds of the general formula (I)

R—O—(CₘH₂ₘO)ₓ—(CₙH₂ₙO)_{y}—R′ (I)

in Which the individual radicals and indices have the following meanings:
R and R′ independently of one another represent hydrogen, a straight-chain C₁- to C₅-alkyl radical or a branched C₃- or C₄-alkyl radical;
m is 2 or 3;
n is or 3;
x is from 5 to 150 and
y is from 5 to 150.

Where one radical n or in has the meaning 2 and the other radical n or m has the meaning 3 as penetrant for water-soluble insecticidally and acaricidally active compounds and/or for increasing the spontaneity when preparing application liquors.

The water-soluble insecticidally and/or acaricidally active compounds for the use according to the invention are preferably selected from the group consisting of compound B, compound A, the group of the neonicotinoids (i.e., imidacloprid, nitenpyram, acetamiprid, thiacloprid, thiamethoxam, clothianidin, dinotefuran), sulfoxaflor and flonicamid.

Particular preference is given to compound B and compound A and the neonicotinoids. Very particular preference is given to compound B and compound A.

In the context of the present invention, a straight-chain C₁- to C₅-alkyl radical is understood as meaning a methyl, ethyl, n-propyl, n-butyl or n-pentyl radical.

In the context of the present invention, a branched C₃- or C₄-alkyl radical is understood as meaning an isopropyl, isobutyl or tert.-butyl radical.

In a preferred embodiment, the, radicals R and R′ are independently of one another selected from the group consisting of a methyl radical, an n-butyl radical and hydrogen.

In an even more preferred embodiment, the radicals R and R′ are independently of one another selected from the group consisting of an n-butyl radical and hydrogen.

With a view to the arrangement of the polyethylene and polypropylene moieties,
(a) either m has the value 2 and n has the value 3;
(b) or m has the value 3 and n has the value 2.

Preferred is embodiment (h) where in is 3 and n is 2.

Very particular preference is given to alkyl polypropylene glycol polyethylene glycol compounds of the formula (I) in Which
m is 3,
n is 2,
x is from 5 to 80,
y is from 5 to 80,
R is n-butyl or hydrogen and
R′ is hydrogen.

According to the invention, it has been found that corresponding compositions generally have penetration-enhancing properties for water-soluble agrochemically active compounds.

According to the invention, it has furthermore been found that corresponding compositions generally increase spontaneity when preparing the application liquor from the concentrate.

According to the invention, it has furthermore been found that corresponding compositions generally have penetration-enhancing properties for water-soluble agrochemically active compounds and increase spontaneity when preparing the application liquor from the concentrate.

For further embodiments of the use according to the invention, reference is made to what was discussed above.

The invention is illustrated by the examples below,

### PREPARATION EXAMPLES

### GENERAL EXAMPLE

To produce a formulation,
20-40 g of alkyl polypropylene glycol polyethylene glycol compound of the formula (I) (the exact amount of additive is stated in Table 1),
43-63 g of solvent (the exact amount is adapted to the amount of alkyl polypropylene glycol polyethylene glycol compound of the formula (I)) and
25 mg of antifoam
   are successively added with stirring at room temperature to
17 g of active compound.

After the addition has ended, the mixture is stirred at room temperature until a transparent solution has formed. This gives a homogeneous liquid.

### Comparative example A

To prepare a formulation,
40 g of emulsifier KS (structure: [RCOO(EO)_{x/y/z}][CH(CH₂)₂]),
43 g of solvent (for example propylene carbonate, 1-methoxy-2-propanol, benzyl alcohol, etc.) and
25 mg of antifoam
   are successively added with stirring at room temperature to
17 g of compound B.

After the addition has ended, the mixture is stirred at room temperature for another 5 minutes. This gives a homogeneous liquid.

### Comparative Example B

To prepare a formulation,
40 g of emulsifier Agnique 3551 (structure: 1-Et-Hex-O—(PO)ₓ-(EO)_{y}—H),
43 g of solvent (for example propylene carbonate, 1-methoxy-2-propanol, benzyl alcohol, etc.) and
25 mg of antifoam
   are successively added with stirring at room temperature to
17 g of compound B.

After the addition has ended, the mixture is stirred at room temperature for another 5 minutes. This gives a homogeneous liquid.

### Comparative Example C

To prepare a formulation,
40 g of emulsifier Agnique KE 3552 (structure: n-Octyl-O—(PO)ₓ-(EO)_{y}—H),
43 g of solvent (for example propylene carbonate, 1-methoxy-2-propanol, benzyl alcohol, etc) and
25 mg of antifoam
   are successively added with stirring at room temperature to
17 g of compound B,

After the addition has ended, the mixture is stirred at room temperature for another 5 minutes. This gives a homogeneous liquid.

### Comparative Example D

To prepare a formulation,
40 g Emulsogen ITA. 200 (structure: iso-C₁₃H₂₇—O(PO)ₓ-(EO)_{y}—H),
43 g of solvent (for example propylene carbonate, 1-methoxy-2-propanol, benzyl alcohol, etc.) and
25 mg of antifoam
   are successively added with stirring at room temperature to
17 g of compound B.

After the addition has ended, the mixture is stirred at room temperature for another 5 minutes. This gives a. homogeneous liquid.

### Comparative Example E

To produce a formulation,
30 g of Antarox B/848 (structure: n-Bu-O-(PO)ₓ-(EO)ₓ-H),
43 g of solvent (for example NMP, hutyrolactone, dimethylimidazolinone, etc.) and
25 mg of antifoam
   are successively added with stirring at room temperature to
8.5 g of flonicamid or sulfoxaflor.

After the addition has ended, the mixture is stirred at room temperature for another 5 minutes. This gives a homogeneous liquid.

### Use Example I

Determination of the spontaneity of a formulation according to the above recipe.

A 100 ml glass cylinder with ml scale (diameter about 3 cm, height about 25 cm) and ground-glass neck is filled to the 100 ml mark with water (CIPAC C) and adjusted to the respective temperature. For the actual test, 1.0 ml of the formulation are added dropwise to the water from a height of 1 cm above the surface of the water, and the behaviour on emersion and sinking is observed. If the drops fall to the bottom, the spontaneity is unsatisfactory; however, if the drops dissolve within the first section of the fall, i.e. within a few cm, the spontaneity is very good. In cases where there is incomplete dissolution on introduction into water, the cylinder is closed with a stopper and inverted up to three times to achieve mixing. The resulting mixture is examined for phase separation (crystalline or oily phases).

The results of such spontaneity tests are summarized in Table I. Remarkable is in particular the observation that the spontaneities of Test Series 2 (with emulsifiers covered inter alia by patent WO 03/090531 A1 mentioned above) are unsatisfactory, whereas the analogous formulations with Antarox B/848 (from Rhodia, Tests 1a to 1c) or Antarox B/500 (from Rhodia, Tests 1d and 1e) or Emulsogen 3510 (from Clariant, Tests 1f and 1g) or Emulsogen EP 4901 (from Clariant, Tests 1h 1i) show good spontaneities. These additives are covered by the definition of the additives to be used according to the invention.

Mixtures of Antarox B/848 and Emulsogen 3510 or Emulsogen EP 4901, too, have good spontaneities (examples 1l to 1q). In addition, Tests 3a to 3c, where the emulsifier Agnique KE 3552 (from Cognis) is used, show, compared to Tests 2a to 2c (emulsifier Agnique KE 3551, from Cognis), that a straight-chain terminal octyl group, compared to a branched group, results in virtually no improvement of spontaneity. Accordingly, it is not the branching but the chain length of the terminal alkyl group that matters, short alkyl groups (or hydrogen) offering advantages with respect to spontaneity properties.

This observation is highly surprising since the alkyl chains are located at the lipophilic (PO) end of the emulsifier and, in spite of their short length, have a very substantial impact on the properties of the emulsifier, compared to the much longer lipophilic (PO) moiety.

Table I: Results of the Spontaneity Tests

Formulations of Compound B with Propylene Carbonate, Benzyl Alcohol or Butyrolactone as Solvent

TABLE I
Formulations (SL-200) of compound B with propylene carbonate as solvent

| | Emulsifier | | | | |
|---|---|---|---|---|---|
| | Concentration, | | SPONTANEITY* | | |
| No. | name | Structure | 10° C. | RT (20° C.) | 30° C. |
| 1a | 40% Antarox B/848 | nBu—O—(PO)ₓ—(EO)_{y}—H | 3.5 | 3 | 3 |
| | according to the | | | | |
| | invention | | | | |
| 1b | 30% Antarox B/848 | nBu—O—(PO)ₓ—(EO)_{y}—H | 3.5 | 2 | 1.5 |
| | according to the | | | | |
| | invention | | | | |
| 1c | 20% Antarox B/848 | nBu—O—(PO)ₓ—(EO)_{y}—H | 4 | 2.5 | 2 |
| | according to the | | (PS) | | |
| | invention | | | | |
| 1d | 40% Antarox B/500 | nBu—O—(PO)ₓ—(EO)_{y}—H | 3 | 2.5 | 2.5 |
| | according to the | | | | |
| | invention | | | | |
| 1e | 30% Antarox B/500 | nBu—O—(PO)ₓ—(EO)_{y}—H | 3 | 2 | 2 |
| | according to the | | | | |
| | invention | | | | |
| 5a | 30% Witconol NS | nBu—O—(PO)ₓ—(EO)_{y}—H | | 1 | |
| | 500 K (Ethylan NS | | | | |
| | 500 LQ) according to | | | | |
| | the invention | | | | |
| 5b | 10% Witconol NS | nBu—O—(PO)ₓ—(EO)_{y}—H | | 4 (PS) | |
| | 500 K (Ethylan NS | | | | |
| | 500 LQ) according to | | | | |
| | the invention | | | | |
| 5c | 30% Atlas G 5000 | nBu—O—(PO)ₓ—(EO)_{y}—H | | 2 | |
| | according to the | | | | |
| | invention | | | | |
| 5d | 10% Atlas G 5000 | nBu—O—(PO)ₓ—(EO)_{y}—H | | 2 (PS) | |
| | according to the | | | | |
| | invention | | | | |
| 5e | 30% Pluronic PE | H—(EO)_{w}—(PO)ₓ—(EO)_{y}—H | | 5 (PS) | |
| | 10500 (Ethylan 324) | | | | |
| 1f | 40% | nBu—O—(PO)ₓ—(EO)_{y}—H | 2.5 | 3 | 3.5 |
| | Emulsogen 3510 | | | | |
| | according to the | | | | |
| | invention | | | | |
| 1g | 20% | nBu—O—(PO)ₓ—(EO)_{y}—H | 4 | 2.5 | 2.5 |
| | Emulsogen 3510 | | (PS) | | |
| | according to the | | | | |
| | invention | | | | |
| 1h | 40% | nBu—O—(PO)ₓ—(EO)_{y}—H | 4 | 3 | 3 |
| | Emulsogen EP 4901 | | (PS) | | |
| | according to the | | | | |
| | invention | | | | |
| 1i | 40% | HO—(PO)ₓ—(EO)_{y}—H | 3.5 | 3 | 4 |
| | Emulsogen V 1816 | | (PS) | | |
| | according to the | | | | |
| | invention | | | | |
| 1j | 20% Antarox B/848, | nBu—O—(PO)ₓ—(EO)_{y}—H, | 3.5 | 3 | 2.5 |
| | 10% Emulsogen V | HO—(PO)ₓ—(EO)_{y}—H | | | |
| | 1816 | | | | |
| | according to the | | | | |
| | invention | | | | |
| 1k | 15% Antarox B/848, | nBu—O—(PO)ₙ—(EO)ₘ—H, | 4 | 3 | 2.5 |
| | 15% Emulsogen V | HO—(PO)ₓ—(EO)_{y}—H | | | |
| | 1816 | | | | |
| | according to the | | | | |
| | invention | | | | |
| 1l | 20% Antarox B/848, | nBu—O—(PO)ₓ—(EO)_{y}—H, | 2.5 | 2.5 | 2 |
| | 10% Emulsogen 3510 | nBu—O—(PO)ₓ—(EO)_{y}—H | (PS) | | |
| | according to the | | | | |
| | invention | | | | |
| 1m | 15% Antarox B/848, | nBu—O—(PO)ₓ—(EO)_{y}—H, | 2.5 | 2 | 1.5 |
| | 15% Emulsogen 3510 | nBu—O—(PO)ₓ—(EO)_{y}—H | | | |
| | according to the | | | | |
| | invention | | | | |
| 1n | 10% Antarox B/848, | nBu—O—(PO)ₓ—(EO)_{y}—H, | 2.5 | 2 | 1.5 |
| | 20% Emulsogen 3510 | nBu—O—(PO)ₓ—(EO)_{y}—H | | | |
| | according to the | | | | |
| | invention | | | | |
| 1o | 20% Antarox B/848, | nBu—O—(PO)ₓ—(EO)_{y}—H, | 3 | 2 | 2 |
| | 10% Emulsogen EP | nBu—O—(PO)ₓ—(EO)ₘ—H | (PS) | | |
| | 4901 | | | | |
| | according to the | | | | |
| | invention | | | | |
| 1p | 15% Antarox B/848, | nBu—O—(PO)ₓ—(EO)_{y}—H, | 2.5 | 2.5 (PS) | 2 |
| | 15% Emulsogen EP | nBu—O—(PO)ₓ—(EO)_{y}—H | (PS) | | |
| | 4901 | | | | |
| | according to the | | | | |
| | invention | | | | |
| 1q | 10% Antarox B/848, | nBu—O—(PO)ₓ—(EO)_{y}—H, | 3 | 2.5 | 2 |
| | 20% Emulsogen EP | nBu—O—(PO)ₓ—(EO)_{y}—H | (PS) | | |
| | 4901 | | | | |
| | according to the | | | | |
| | invention | | | | |
| 2a | 40% Agnique 3551 | Et—Hex—O—(PO)ₓ—(EO)_{y}—H | 6 | 6 (PS) | 6 (PS) |
| | comparative | | (PS) | | |
| 2b | 30% Agnique 3551 | Et—Hex—O—(PO)ₓ—(EO)_{y}—H | 6 | 3 (PS) | 3 (PS) |
| | comparative | | (PS) | | |
| 2c | 20% Agnique 3551 | Et—Hex—O—(PO)ₓ—(EO)_{y}—H | 6 | 3 (PS) | 3 (PS) |
| | comparative | | (PS) | | |
| 2d | 40% | Iso—C₁₃H₂₇—O—(PO)ₓ—(EO)_{y}—H | 6 | 6 (PS) | 5 (PS) |
| | Emulsogen ITA 200 | | (PS) | | |
| | comparative | | | | |
| 2e | 40% | RO—(PO)ₓ—(EO)_{y}—H | 6 | 5 (PS) | 4 (PS) |
| | Genapol EP 2584 | R = long-chain | (PS) | | |
| | comparative | alcohol (not | | | |
| | | according to the | | | |
| | | invention) | | | |
| 3a | 40% | n-Oct—O—(PO)ₓ(EO)_{y}—H | 6 | 3 (PS) | 3 (PS) |
| | Agnique KE 3552 | | (PS) | | |
| | comparative | | | | |
| 3b | 30% | n-Oct—O—(PO)ₓ—(EO)_{y}—H | 6 | 3 (PS) | 3 (PS) |
| | Agnique KE 3552 | | (PS) | | |
| | comparative | | | | |
| 3c | 20% | n-Oct—O—(PO)ₓ—(EO)_{y}—H | 6 | 3 (PS) | 3 (PS) |
| | Agnique KE 3552 | | (PS) | | |
| | comparative | | | | |
| 4 | 40% | [RCOO(EO)_{x/y/z}][CH(CH₂)₂] | 6 | 6 (PS) | 6 (PS) |
| | Emulgator KS | | (PS) | | |
| | comparative | | | | |

*Evaluation scale 1-6, i.e. 1 = very good, 6 = insufficient; PS = phase separation; RT = room temperature (20° C.), x, y, z, n, m are integers ≧2.

TABLE II
Formulations (SL-200) of compound B with different solvents

| | | Emulsifier | | | | |
|---|---|---|---|---|---|---|
| | | Concentration, | | SPONTANEITY* | | |
| No. | Solvent | Name | Structure | 10° C. | RT (20° C.) | 30° C. |
| | Propylene carbonate | No emulsifier | | 6 (PS) | 6 (PS) | 6 (PS) |
| 1a | | 40% Antarox | nBu—O—(PO)ₓ—(EO)_{y}—H | 3.5 | 3 | 3 |
| | | B/848 | | | | |
| | | according to the | | | | |
| | | invention | | | | |
| 1b | | 30% Antarox | nBu—O—(PO)ₓ—(EO)_{y}—H | 3.5 | 2 | 1.5 |
| | | B/848 according to | | | | |
| | | the invention | | | | |
| 1c | | 20% Antarox | nBu—O—(PO)ₓ—(EO)_{y}—H | 4 (PS) | 2.5 | 2 |
| | | B/848 according to | | | | |
| | | the invention | | | | |
| 3b | | 30% | n-Oct—O—(PO)ₓ—(EO)_{y}—H | 6 (PS) | 3 (PS) | 3 (PS) |
| | | Agnique KE 3552 | | | | |
| | | comparative | | | | |
| | Benzyl alcohol | No emulsifier | | 6 (PS) | 6 (PS) | 6 (PS) |
| | | 40% Antarox | nBu—O—(PO)ₓ—(EO)_{y}—H | 4 | 4 | 4 |
| | | B/848 | | | | |
| | | according to the | | | | |
| | | invention | | | | |
| | | 30% Antarox | nBu—O—(PO)ₓ—(EO)_{y}—H | 4 | 5 (PS) | 6 (PS) |
| | | B/848 according to | | | | |
| | | the invention | | | | |
| | | 20% Antarox | nBu—O—(PO)ₓ—(EO)_{y}—H | 6 (PS) | 6 (PS) | 6 (PS) |
| | | B/848 according to | | | | |
| | | the invention | | | | |
| | | 30% | n-Oct—O—(PO)ₓ—(EO)_{y}—H | 6 (PS) | 6 (PS) | 6 (PS) |
| | | Agnique KE 3552 | | | | |
| | | comparative | | | | |
| | Benzyl alcohol/ | No emulsifier | | 6 (PS) | 6 (PS) | 6 (PS) |
| | □-butyrolactone (1:1) | 40% Antarox | nBu—O—(PO)ₓ—(EO)_{y}—H | 3 | 2 | 2 |
| | | B/848 | | | | |
| | | according to the | | | | |
| | | invention | | | | |
| | | 30% Antarox, | nBu—O—(PO)ₓ—(EO)_{y}—H | 1 | 1 | 1 |
| | | B/848 according to | | | | |
| | | the invention | | | | |
| | | 20% Antarox | nBu—O—(PO)ₓ—(EO)_{y}—H | 1 | 1 | 1 |
| | | B/848 according to | | | | |
| | | the invention | | | | |
| | | 30% | n-Oct—O—(PO)ₓ—(EO)_{y}—H | 4 | 5 | 5 |
| | | Agnique KE 3552 | | | | |
| | | comparative | | | | |

*Evaluation scale 1-6, i.e. 1 = very good, 6 = insufficient; PS = phase separation

TABLE III
Acetamiprid formulation with benzyl alcohol as solvent

| | Emulsifier | | SPONTANEITY* | | |
|---|---|---|---|---|---|
| No. | Name | Structure | 10° C. | RT (20° C.) | 30° C. |
| | 30% Antarox B/848 | nBu—O—(PO)ₓ—(EO)_{y}—H | 2 | 2 (clear) | 2 |
| | | | (clear) | | (clear) |

TABLE IV
Flonicamid formulations with NMP as solvent

| | Emulsifier | | SPONTANEITY* | | |
|---|---|---|---|---|---|
| No. | Name | Structure | 10° C. | RT (20° C.) | 30° C. |

| | | |
|---|---|---|
| 30% Agnique 3551 | Et—Hex—O—(PO)ₓ—(EO)_{y}—H | 2.5 |
| comparative | | (turbid) |
| 30% Antarox B/848 | nBu—O—(PO)ₓ—(EO)_{y}—H | 2 (clear) |
| according to the | | |
| invention | | |
| 30% Antarox B/500 | nBu—O—(PO)ₓ—(EO)_{y}—H | 2 (clear) |
| according to the | | |
| invention | | |

TABLE V
Sulfoxaflor formulations with dimethylimidazolinone (DMI) as solvent

| | Emulsifier | | SPONTANEITY* | | |
|---|---|---|---|---|---|
| No. | Name | Structure | 10° C. | RT (20° C.) | 30° C. |

| | | |
|---|---|---|
| 30% Agnique 3551 | Et—Hex—O—(PO)ₓ—(EO)_{y}—H | 2.5 |
| comparative | | (turbid) |
| 30% Antarox B/848 | nBu—O—(PO)ₓ—(EO)_{y}—H | 2 (clear) |
| according to the | | |
| invention | | |

TABLE VI
Formulations of compound A with propylene carbonate as solvent

| | Emulsifier | | SPONTANEITY* | | |
|---|---|---|---|---|---|
| No. | Name | Structure | 10° C. | RT (20° C.) | 30° C. |

| | | |
|---|---|---|
| 30% Agnique 3551 | Et—Hex—O—(PO)ₓ—(EO)_{y}—H | 3.5 (PS) |
| comparative | | |
| 30% Antarox B/848 | nBu—O—(PO)ₓ—(EO)_{y}—H | 2.5 |
| according to the | | |
| invention | | |
| 20% Antarox B/848 | nBu—O—(PO)ₓ—(EO)_{y}—H | 3 |
| according to the | | |
| invention | | |

### Use Example II

Determination of the penetration properties of a formulation according to the above recipe.

This test measures the penetration of active compounds (a.i.) by enzymetically isolated cuticles from apple tree leaves.

Fully developed leaves cut from apple trees of the Golden Delicious variety are used. The cuticles are isolated by
initially filling, by vacuum infiltration, leaf discs which had been marked on the underside with dye and punched out, with a pectinase solution (0.2 to 2% strength) buffered to a. pH between 3 and 4,
then adding sodium azide and
allowing the leaf discs treated in this manner to stand until the original leaf structure has been dissolved and the non-cellular cuticles have been detached.

Subsequently, only the cuticles free from stomata and hairs of the upper sides of the leaves are used. They are washed repeatedly, alternating with water and a buffer solution of pH 7. The resulting clean cuticles are then mounted on Teflon plates and straightened and dried using a gentle stream of air.

In the next step, the cuticle membranes obtained in this manner are, for membrane transport studies, placed into diffusion cells (=transport chambers) made from stainless steel. To this end, the cuticles are, using a pair of tweezers, placed into the middle of the edges of the diffusion cells, which have been coated with silicone fat, and the cuticles are closed using a ring which had also been treated With fat. The arrangement is chosen such that the morphological outside of the cuticles is facing out, i.e. to the atmosphere, whereas the original inside is facing the inside of the diffusion cell. The diffusion cells are filled with water or a water/solvent mixture.

To determine penetration, in each case 5 μl of an active compound-comprising application liquor of the formulations mentioned below are applied to the outside of a cuticle. The active compound concentration corresponds to the outer application rate customary in practice.

In each case, tap water is used for the application liquors.

After the application of the application liquors, the water is in each case allowed to evaporate, and the chambers are then inverted and placed into thermostatic taps, where in each case air of a defined atmospheric humidity and temperature is blown onto the outside of the cuticles. Accordingly, the penetration that sets in takes place at a relative atmospheric humidity of 60% and a temperature adjusted to 25° C., Using an autosampler, samples are taken at regular intervals, and the content of penetrated active compound is measured.

The test results for various active compounds can be seen in the examples below. The stated numbers are average values of in each case 10 individual measurements.

### Example I

### Penetration of acetamiprid (0.5 g of a.i./I) through Apple Leaf Cuticles

| | Penetration after |
|---|---|
| Application liquor | 24 h (in %) |
| a.i. in water | 61 |
| a.i. in water & Antarox B/848 | 100 |
| a.i. in water & Antarox B/500 | 100 |
| Comparative example 1: a.i. in water & | 82 |
| emulsifier W (styrenated α-phenyl-ω- | |
| hydroxylpolyoxyethane-1,2-diyl, CAS | |
| 104376-72-9) | |

### Example II

### Penetration of Compound A (0.5 g of a.i./I) through Apple Leaf Cuticles

| | Penetration after |
|---|---|
| Application liquor | 24 h (in %) |
| a.i. in water | 2 |
| a.i. in water & Antarox B/848 | 49 |
| Comparative example 1: a.i. in water & | 7 |
| emulsifier W (styrenated α-phenyl-ω- | |
| hydroxylpolyoxyethane-1,2-diyl, CAS | |
| 104376-72-9) | |

### Example III

### Penetration of Compound B (0.3 g of a.i./I) through Apple Leaf Cuticles

| | Penetration after |
|---|---|
| Application liquor | 24 h (in %) |
| Labor-EC & Antarox B/848, 0.3 g/l | 23 |
| Labor-EC & Antarox B/848, 0.6 g/l | 64 |
| Comparative example 1: Aqueous | 18 |
| application liquor consisting of 0.5 g/l of | |
| active compound BYI 02960, 0.5 g/l | |
| emulsifier W (styrenated α-phenyl-ω- | |
| hydroxylpolyoxyethane-1,2-diyl, CAS | |
| 104376-72-9) and 20 g/l dimethylacetamide | |

### Example IV

### Penetration of Adjuvant-Comprising SL Formulations of Compound B (0.3 g of a.i./I) through Apple Leaf Cuticles

| | Penetration after |
|---|---|
| Spray liquor | 24 h (in %) |
| SL 200 (2008-000189) with Antarox | 72 |
| B/848 (40%) | |
| Comparative example: SL 300 (without | 10 |
| adjuvant) | |

More active compound was used in the comparative example than in the spray liquor according to the invention, so that an increased penetration would have been assumed for the comparative example.

### Example V

### Penetration of Compound B (03 g of a.i./I) through Apple Leaf Cuticles

| | Penetration after |
|---|---|
| Spray liquor | 24 h (in %) |
| a.i. in water | 4 |
| a.i. in water & Antarox B/848, 1 g/l | 48 |
| a.i. in water & Antarox B/500, 1 g/l | 52 |
| Comparative example 1: | 13 |
| a.i. in water & emulsifier W, 1 g/l | |

### Use Examples III

Determination of the retention of spray liquors

Retention is to be understood as meaning the adherence of spray mists to surfaces, for example plants or plant parts (leaves), after spray application. The retention depends both on the composition of the spray liquor and on the nature of the treated surface,

Routinely, the retention of a spray liquor is determined using a poorly wettable leaf in comparison to a reference surface with 100% retention, The reported results were obtained using primary leaves of barley seedlings which had been cultivated in a greenhouse (18° C., 80% atmospheric humidity, 16 h of light).

Individual cut leaves are subjected to standard treatment in a spray cabin using a track sprayer—nozzle: XR 11002 VS; pressure: 3 bar; 300 l/ha. Weight and leaf area are determined prior to the treatment, and the adhering spray coating is correspondingly determined gravimetrically and normalized to the leaf area. A nonwoven-fabric; paper cloth serves as reference surface for 100% retention with a precisely known area,

Per spray liquor variant, at least 5, better 8-10, individual leaves are treated, and the calculated retentions are then averaged. In general, a test series with pure tap water is carried out as negative control (lowest retention).

In such a comparative test, spray liquors with the additive Antarox B/848 (from Rhodia, chemical structure n-Bu—O—(PO)ₓ-(EO)_{y}—H) or Agnique 3551 (from. Cognis, chemical structure 2-ethyl-hexyl-O—(PO)ₓ-(EO)_{y}—H) or Agnique KE 3552 (from Cognis, chemical structure n-Oct-O—(PO)ₓ-(EO)_{y}—H) all gave excellent and, within the accuracy of the method, equal retention values.

This Test Series III confirms that, in spite of improved penetration and improved spontaneity, other properties, in particular retention, are not adversely affected.

### Use Examples IV

### Comparison of the Biological Activity

### Translaminar Action of Compound B Against Myzus persicae on Cabbage Leaves

Cabbage leaves are infected with a mixed population of the green peach aphid (Myzus persicae). Once the aphids have colonized the underside of the leaf, the plants are sprayed with the substances listed in the table using a bar sprayer such that only the upper side of the leaf is treated with insecticide (volume of water: 300 l/ha). The translaminar efficacy of the treatment against a mixed population of the green peach aphid in % mortality was determined at the stated points in time:

| | | Translaminar action against | |
|---|---|---|---|
| | Active | Myzus persicae | |
| | compound | [% mortality] | |
| | application | on cabbage | |
| | rate | 2 days after | 6 days after |
| Product | [g/ha] | application | application |
| Compound B | 120 | 0 | 20 |
| SL 200 | 60 | 0 | 0 |
| without addition of | 24 | 0 | 0 |
| Antarox B/848 | 4.8 | 0 | 0 |
| | 0.96 | 0 | 0 |
| | 0.192 | 0 | 0 |
| Compound B | 120 | 90 | 100 |
| SL 200 | 60 | 40 | 80 |
| with addition of Antarox | 24 | 0 | 20 |
| B/848 in the spray liquor | 4.8 | 0 | 0 |
| (about 0.6 g per gram | 0.96 | 0 | 0 |
| of compound B) | 0.192 | 0 | 0 |
| Compound B | 120 | 94 | 100 |
| SL 200 | 60 | 65 | 90 |
| with addition of Antarox | 24 | 0 | 30 |
| B/848 in the spray liquor | 4.8 | 0 | 0 |
| (2.9 g per gram of | 0.96 | 0 | 0 |
| compound B) | 0.192 | 0 | 0 |

1. Composition, comprising at least one active compound selected from the group consisting of compound B and compound A and at least one alkyl polypropylene glycol polyethylene glycol compound of the general formula (I)
R—O—(CₘH₂ₘO)ₓ—(CₙH₂ₙO)_{y}—R′ (I)
in which the individual radicals and indices have the following meanings:
R and R′ independently, of one another represent hydrogen, a straight-chain C₁- to C₅-alkyl radical or a branched C₃- or C₄-alkyl radical;
m is 2 or 3;
n is 2 or 3;
x is from 5 to 150 and
y is from 5 to 150,
where one radical n or m has the meaning 2 and the other radical n or m has the meaning 3 and where compound B has the formula 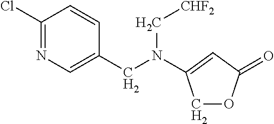
and compound A has the formula

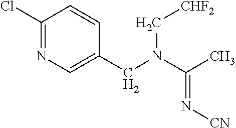

2. Composition according to claim 1, characterized in that the radicals R and R′ are independently of one another selected from the group consisting of a methyl radical, an n-butyl radical and hydrogen.

3. Composition according to claim 1, characterized in that the radicals R and R′ are independently of one another selected from the group consisting of an n-butyl radical and hydrogen.

4. Composition according to claim 1, characterized in that in the alkyl polypropylene glycol polyethylene glycol compound of the formula (I)
m is 3,
n is 2,
x is from 5 to 80,
y is from 5 to 80,
R is n-butyl or hydrogen and
R′ is hydrogen.

5. Composition according to claim 1, characterized in that it furthermore comprises, as agrochemically active compound, imidacloprid, nitenpyram, acetamiprid, thiacloprid, thiamethoxam, clothianidin, dinotefuran, sulfoxaflor and/or flonicamid.

6. Composition according to claim 1, characterized in that the composition comprises from 0.1 to 80% by weight of the alkyl polypropylene glycol polyethylene glycol compound of the general formula (I), based on the composition.

7. Composition according to claim 1, in the form of a concentrate.

8. Process for preparing a composition according to claim 1, characterized in that the individual components are mixed with one another in the respective desired ratios.

9. Process according to claim 8, characterized in that an agrochemically active compound is initially charged and the other components are then added to the composition with stirring, in any order.

10. Use of a composition according to claim 1 in the agrochemical field.

11. Use according to claim 10, characterized in that the composition is used for controlling insects and/or Acarina.

12. Use according to claim 10, where a composition according to claim 6 is initially converted into a spray liquor and the resulting spray liquor is used.

13. Use of alkyl polypropylene glycol polyethylene glycol compounds of the general formula (I)
R—O—(CₘH₂ₘO)ₓ—(CₙH₂ₙO)_{y}—R′ (I)
in which the individual radicals and indices have the following meanings:
R and R′ independently of one another represent hydrogen, a straight-chain C₁- to C₅-alkyl radical or a branched C₃- or C₄-alkyl radical;
m is 2 or 3;
n is 2 or 3;
x is from 5 to 150 and
y is from 5 to 150,
where one radical n or m has the meaning 2 and the other radical n or m has the meaning 3 as penetrants for water-soluble insecticidally and/or acaricidally active compounds and/or for enhancing the spontaneity of preparations when preparing application liquors.

14. Use according to claim 13, characterized in that the radicals R and R′ are independently of one another selected from the group consisting of a methyl radical, an n-butyl radical and hydrogen.

15. Use according to claim 13 as penetrant for water-soluble insecticides and/or acaricides where these are added as a tank mix to an application liquor and/or alkyl polypropylene glycol polyethylene glycol compounds are added to a provisional application liquor.
